(19) United States
(12) Patent Application Publication
(10) Pub. No.: US 2022/0192810 A1
(43) Pub. Date: Jun. 23, 2022

(54) DEVICE FOR THE MANAGEMENT OF INCONTINENCE-RELATED SYMPTOMS

(71) Applicant: Fannin Partners LLC, Houston, TX (US)

(72) Inventors: Leo LINBECK, III, Houston, TX (US); Michael John Heffernan, Katy, TX (US); Deepti Gopinath, Houston, TX (US); Christine Luk, Houston, TX (US); Carine Rizk, Houston, TX (US); Amanda Gibbens, Houston, TX (US)

(21) Appl. No.: 17/557,970

(22) Filed: Dec. 21, 2021

Related U.S. Application Data
(60) Provisional application No. 63/128,216, filed on Dec. 21, 2020.

(51) Int. Cl. A61F 2/00

(52) U.S. Cl. CPC A61F 2/0036; A61F 2/0009

(57) ABSTRACT

The present invention relates to the application of medical devices that manage urinary incontinence. The inter-bladder/urethra implanted device allows for passive urine leakage above a threshold bladder pressure and subject-controlled opening and closing of a valve to allow urine passage, below the threshold bladder pressure. The valve is preferably a deformable sac which seals against the interior of the urethra when closed. The valve can be opened by withdrawing slightly from the urethra surface. Passive urine leakage to prevent over-pressure can be facilitated by including external channels on the valve sac. In another embodiment, the valve sac has a central passage which expands and contracts for opening and closing, respectively.

### BACKGROUND

In the United States, 51.1% of women and 13.9% of men suffer from some form of urinary incontinence. The main types of urinary incontinence are stress, urge, overflow, functional, mixed, and reflex incontinence. Many of these are caused by neurogenic bladder: a condition with lack of bladder control due to neurological damage, spinal cord injury, or other disease. Less severe incontinence can be managed by using absorbent pads or minor surgery, but severe incontinence requires more involved surgical procedures and/or frequent catheterization along with medication. Neurogenic bladder can involve loss of function in the muscles of the urethral sphincter, resulting in constant or intermittent leakage of urine from the bladder. Due to the lack of control over the bladder musculature, it can also lead to the inability to void; thus allowing a large amount of urine to build up in the bladder, often causing over-pressurization of the bladder. When the bladder is significantly over-pressurized the change from the normal pressure gradient between the bladder and kidneys can cause a severe reduction in urine flow from the kidneys to the bladder, which can result in renal damage or failure.

The current standard of care for these patients is to take anticholinergic drugs, such as oxybutynin, to relax the bladder musculature, as well as to perform intermittent catheterization 4 to 5 times per day to void. In many of these patients, oxybutynin and intermittent catheterization are not sufficient to prevent urine leakage. These patients may then consider surgical treatments; however, these procedures are highly invasive and are not effective in all patients. At present there is no ideal, non-invasive method to control urinary flow and prevent leakage.

### SUMMARY

The invention relates to the application of medical devices that manage urinary incontinence. The intra-bladder/urethra implanted device allows for passive urine leakage above a threshold bladder pressure and subject-controlled opening and closing of a valve to allow urine passage, below the threshold bladder pressure.

The passive leakage of urine above the threshold bladder pressure is to ensure that the bladder does not over-pressurize and cause damage to the kidneys. The bladder is generally considered to be over-pressured (where there is risk of kidney damage) starting between about 35 cm H2O and 40 cm H2O. This passive leakage is a key feature in that patients can thus safely control urine release at convenient intervals, when the bladder pressure is below the threshold bladder pressure, to thereby prevent over-pressure leakage from occurring.

Preferably, the valve in the device is “closed” in the default position, where a deformable valve sac forms a seal with the urethra and urine does not leak from the bladder. The valve should move between open and closed positions smoothly as the subject actuates the valve with an external remote-control device. Preferably, the device can be resized in order to fit in different urethral anatomies, including for pediatric or adult use, or as the patient urethra grows. Ideally, the sizing should be adjustable by the physician or surgeon while the valve resides in the urethra (i.e. without removal of the device).

Several different embodiments of the device and means for controlling it are depicted in the following figures and the detailed description.

### BRIEF DESCRIPTION OF THE DRAWINGS

Note: In the figure descriptions, the “upper” end of the device, as relates to elevational and plan views, is the end that is closest to the bladder (the proximal end).

FIG. 1A is a cross-sectional view of the urinary valve device in the “open” position, depicted as situated in the bladder neck and urethra.

FIG. 1B is a cross-sectional view of the urinary valve device in the “closed” position, depicted as situated in the bladder neck and urethra.

FIG. 1C is an elevational view of the urinary valve device in the “closed” position.

FIG. 1D is a plan view of the urinary valve device of FIG. 1C (in the “closed” position) from the uppermost side.

FIG. 1E is a plan view of the urinary valve device of FIG. 1C (in the “closed” position) from the lowermost side.

FIG. 1F is a plan view of the urinary valve device of FIG. 1C (in the “open” position) from the uppermost side.

FIG. 1G is a plan view of the urinary valve device of FIG. 1C (in the “open” position) from the lowermost side.

FIG. 2A is a perspective view of one embodiment of a linear displacement mechanism to control opening and closing of an implantable urinary valve device in accordance with the invention.

FIG. 2B is an elevational, expanded view of the nut shown in FIG. 2A.

FIG. 3A is a cross-sectional view of an embodiment of the system used to make fine adjustments to the diameter when the valve is in the closed position.

FIG. 3B is an elevational view of the device showing notches in the screw for making make fine adjustments to the diameter when the valve is in the closed position.

FIG. 4 depicts the user interface screen in a remote-control device for opening and closing the urinary valve.

FIG. 5A is a cross-sectional view of a different embodiment of a urinary valve device, having a central bore, in the “closed” position, depicted as situated in the bladder neck and urethra.

FIG. 5B is a cross-sectional view of the embodiment of FIG. 5A, in the “open” position, depicted as situated in the bladder neck and urethra.

FIG. 6A is a plan view of the embodiment of FIG. 5A, in the “closed” position.

FIG. 6B is a plan view of the embodiment of FIG. 5A, in the “open” position.

FIG. 7A is a cross-sectional view of a modified embodiment similar in principle to the urinary valve device shown in FIG. 5A, depicted in the “closed” position and as situated in the bladder neck and urethra.

FIG. 7B is a cross-sectional view of the embodiment of the urinary valve device shown in FIG. 7A in the “open” position.

FIG. 8A depicts a cross-sectional side-view of the urethra and bladder with a portion of the urethra surgically removed.

FIG. 8B depicts a cross-sectional view of the urethra following removal of a portion as in FIG. 8A and addition of a supporting tube, with a perspective view of a valve of FIGS. 1A, 1B in place in the closed position.

FIG. 8C depicts a cross-sectional view of the urethra following removal of a portion as in FIG. 8A and addition of a supporting tube, with a perspective view of a valve of FIGS. 1A, 1B in place in the open position.

FIG. 8D depicts a cross-sectional view of the urethra with addition of a supporting tube, with a perspective view of a valve of FIGS. 1A, 1B in place in the closed position.

FIG. 9A is a plan view of the portion of the valve device in FIGS. 1C to 1G responsible for sealing with the urethra wall or supporting tube, showing outer channels.

FIG. 9B is an elevational view of the portion of the valve device in FIGS. 1C to 1G responsible for sealing with the urethra wall or supporting tube, showing outer channels.

FIG. 10 depicts a functional block diagram of the drive mechanism and electromechanical components of the valve device.

### DETAILED DESCRIPTION

Unless defined otherwise, all technical and scientific terms used herein have the same meaning as commonly understood by one of the ordinary skills in the art to which this invention belongs. Although any methods and materials similar or equivalent to those described herein can be used in the practice or testing of the present invention, the preferred methods and materials are described.

As used herein, each of the following terms has meaning associated with it in this section. The articles “a” and “an” are used herein to refer to one or to more than one (i.e. to at least one) of the grammatical object of the article. By way of example, “an element” means one element or more than one element.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual members within that range, for example, 1, 2, 2.7, 3, 4, 5, 5.3, and 6. This applies regardless of the breadth of the range.

Valve Sac—refers to a material of any size and composition that optionally envelops a fluid of any type, composition or viscosity, including gases and/or liquids.

Elastomeric Material or Elastomeric Membrane—refers to a silicone material, a rubber, or any other material that has the required elastic properties.

Viscoelastic Material—refers to a material with viscoelasticity, including an amorphous polymer, a semicrystalline polymer, or a biopolymer.

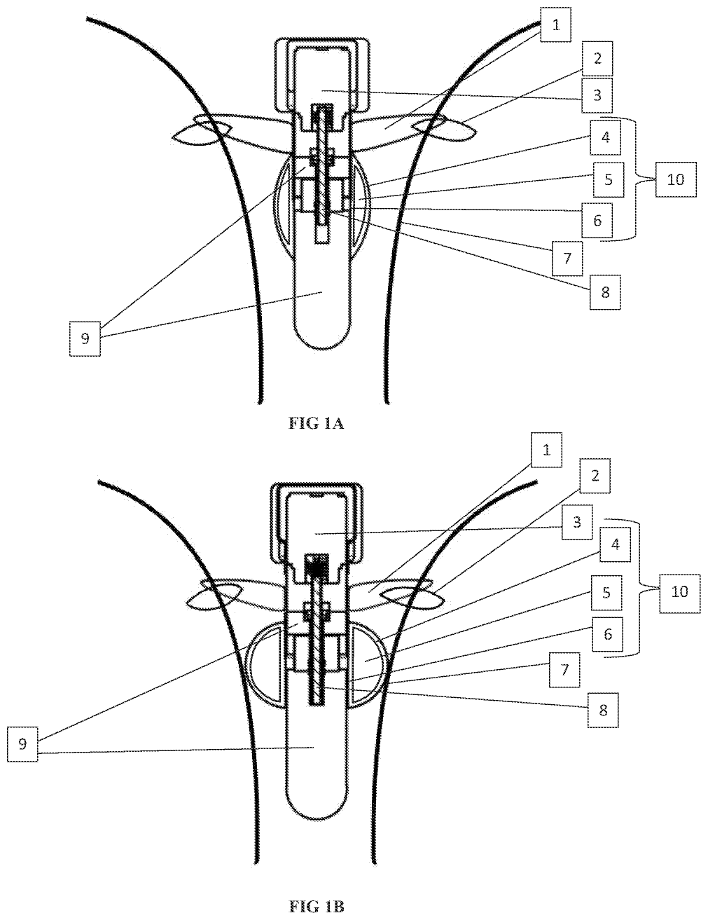

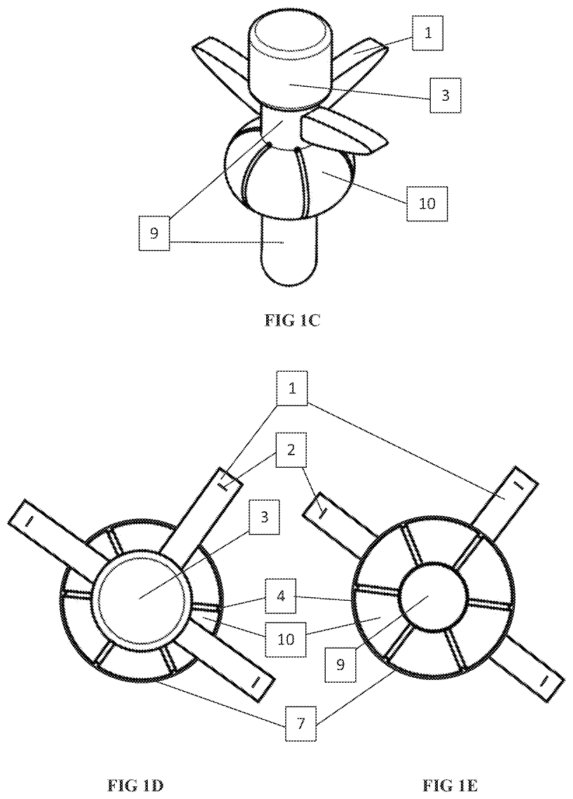

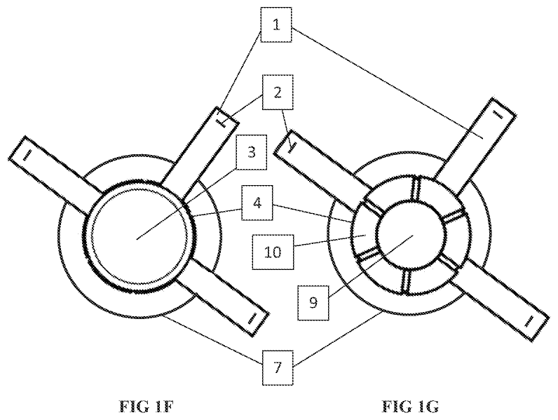

Referring to FIGS. 1A to 1G, a preferred embodiment of the urinary valve device is shown in both the “open” and “closed” settings. The urinary valve device consists of a torque mechanism 3 which, when actuated by an external remote-control (FIG. 4), causes the drive mechanism 8 to rotate. The rotation of the drive mechanism separates the two parts of valve body 9, moving one part away from the neck of the bladder, by converting the rotational motion to linear displacement. This linear displacement causes the valve to move from the “open” position in FIG. 1A to the closed position in FIGS. 1B-1G. The valve body 9 has a length such that it preferably spans from the bladder neck to the upper portion of the urethra. The top of the torque mechanism is attached to flanges (which can be flaps) 1, that anchor to the bladder wall with anchor mechanisms, which can include sutures, clamps, and hooks 2. The flanges 1 can be movable and allow for the anchor mechanisms to be easily implanted within the subject's urinary bladder walls by anchoring to their larger surface(s).

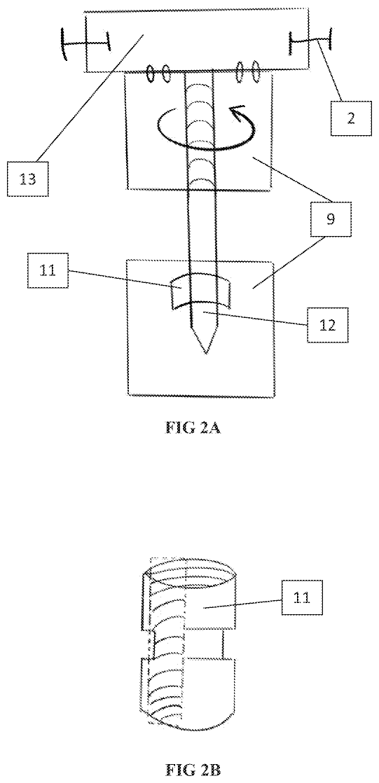

Referring to FIGS. 1A, 1B, 2A, and 2B, the torque mechanism 3, valve body 9, and drive mechanism 8 all lie within valve sac 10, which can be a viscoelastic material or an elastomer shell of appropriate material, having wall thickness (up to an including solid throughout) to allow appropriate deformation under the fluid pressure in the bladder. Alternatively, valve sac 10 can be an impermeable membrane which houses a viscoelastic substance 5. In the latter case, valve sac 10 preferably also houses an inner elastic sac 6 which isolates all portions of the torque mechanism 3 (including, valve body 9, drive mechanism 8, motor 13, lead screw 12, nut 11 from the viscoelastic substance 5, as well as an outer elastic sac 4 which isolates the viscoelastic substance from the urine and tissue. The inner elastic sac 6 and/or outer elastic sac 4 preferably extend around the entire device to isolate the device components from the urine and tissue. The viscoelastic substance 5 is preferably a biocompatible fluid that is in a liquid state at internal body temperature; or optionally, a gas.

The outer diameter of the valve sac 10 varies based on the state of the device. In the open state it is smaller, allowing urine to flow easily around its sides. In the closed state, the outer surface of valve sac 10 expands and forms a seal against the region of the bladder neck and urethral wall 7. Referring to FIGS. 1A to 1G, when the torque mechanism 3 is actuated, the two parts of the valve body 9 move apart causing valve sac 10 to also elongate, as shown in FIG. 1A, leading to a decrease in the outer diameter of valve sac 10. This decrease breaks the seal between valve sac 10 and urethral wall and allows urine to flow along the sides of valve sac 10 and out the urethra.

Referring to FIGS. 1A, 1B, 2A, 2B, 3A and 3B, in the preferred embodiment, drive mechanism 8 will be substantially concentric with valve body 9. The torque mechanism 3 can include a motor 13, powered by a battery, which is preferably rechargeable. Torque mechanism 3 preferably further includes a lead screw 12 that can function as drive mechanism 8 where a nut 11 with mating threads to those of lead screw 12 is fixed within valve body 9. In another embodiment, the motor 13 can be replaced with a cylindrical magnet arrangement capable of rotating and driving drive mechanism 8, or with a solenoid which acts to separate the two parts of the valve body 9. In other embodiments, drive mechanism 8 may include a ratchet system for gears, and the gears may include worm gears or a rack and pinion, or other drive mechanisms. In yet other embodiments, drive mechanism 8 can be operated by moving external (ex vivo) magnets, wherein at least one of the two parts of valve body 9 is itself magnetic or paramagnetic and can be moved by magnetic induction and/or attraction or repulsion.

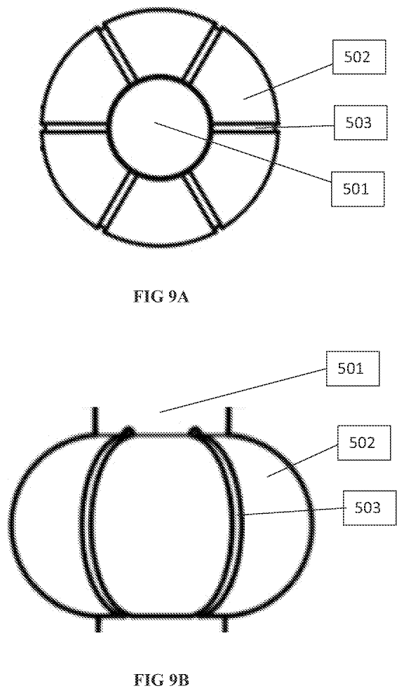

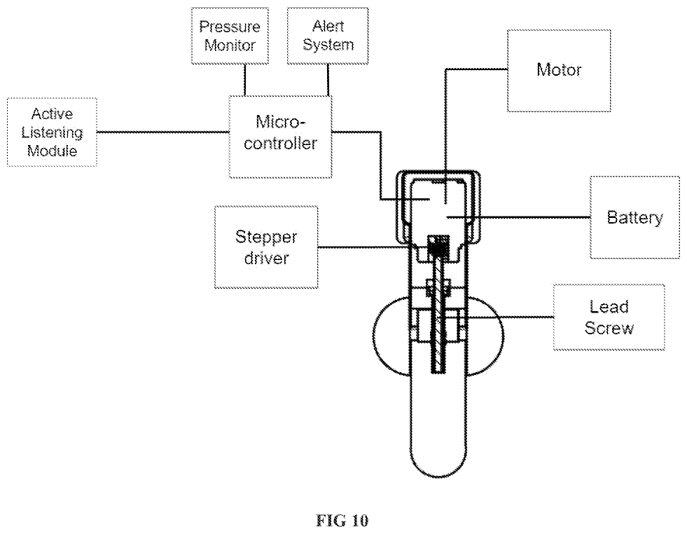

In FIG. 1B the urinary valve device is shown in place in the closed position. The valve sac 10 forms a seal with the interior surface of the urethra 7, preventing any urine from passing through the urethra 7. However, if the bladder pressure is above the threshold pressure, the pressure the urine exerts on the valve sac 10 causes it to deform slightly. This elastic deformation allows the seal to break and for urine to leak out between the valve sac 10 and urethra 7. The urine leakage continues until the bladder achieves pressures lower than the threshold pressure, at which point the elastic deformation is reversed and the valve sac 10 again forms a seal with the urethra 7. The seal will remain in place unless the bladder is over-pressurized again or external control is used to open the device. Channels 503 (FIGS. 9A, 9B) may lie on the outer surface of valve sac 10.

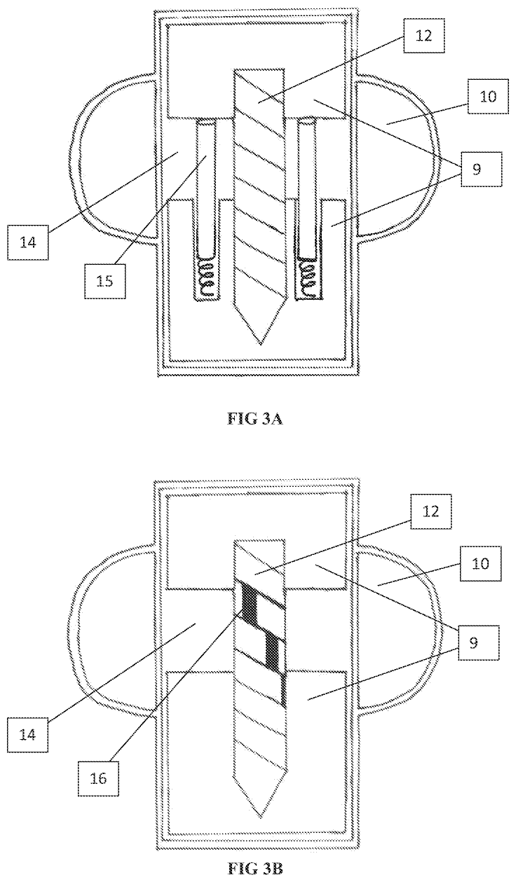

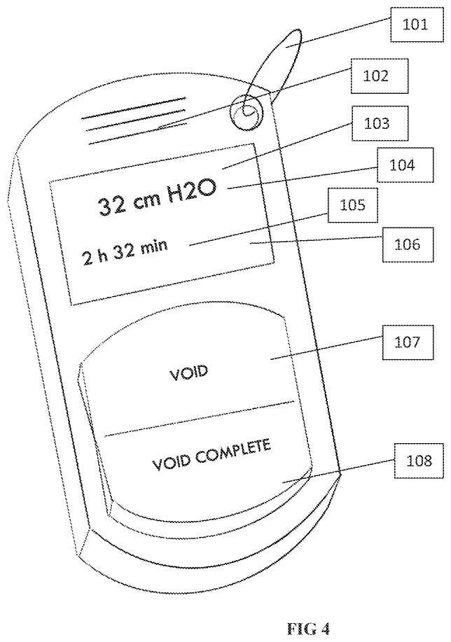

Referring to FIGS. 3A, 3B: to ensure that the diameter of the valve sac 10 in its closed state matches the urethral anatomy of the patient, the diameter of the valve sac 10 can be adjusted to ensure a seal, by including, for example, one or more spring-loaded spacers 15 (FIG. 3A) or providing ridges or notches 16 (FIG. 3B) along the threads of lead screw 12. Other spacers such as a solid block could also be placed between the two parts of valve body 9. The surgeon implanting the device could add the spacers 15 or the extent of drawing together the two parts of valve body during implantation. Spacers prevent the valve body 9 from closing completely, and instead create a space 14 in the middle of the valve body (i.e., valve body remains partially extended). Due to the partial extension of the valve body, the valve sac 10 is stretched and has a smaller diameter at the state of rest of motor 13, in comparison to when there is little or no space 14.

In an embodiment with no spacers, drive mechanism 8 can be back driven by compression of elastic membranes 4 and 6, thereby causing the valve sac 10 to press too tightly against the urethra. Such back driving can be prevented by any of: (i) program the motor 13 to stop at a position where the two parts of valve body 9 are adequately spaced to prevent urine leakage up to a threshold pressure; (ii) adding ridges or notches 16 on lead screw 12 (as shown in FIG. 3B), nut 11, or valve body 9 prevents the two parts of valve body 9 from moving together more than a specified spacing. When utilized, the ridges or notches 16 can be on lead screw 12 or elsewhere on drive on mechanism 8, such that one creates a “high-friction zone” on lead screw 12 as it returns to the closed, resting position. If motor 13 is turned off before reaching the end of travel, the two parts of valve body 9 remain at their positions. If a “more closed” (larger outer diameter for valve sac 10) position is desired, motor 13 drives mechanism 8 through the high-friction zone to reach it. With this type of closing, the resting positions of the two parts of valve body 9 can be set and adjusted remotely, following implantation of the device.

FIG. 4 depicts a preferred embodiment of the remote-control device used to actuate the urinary valve from closed to open and vice versa. When the patient wishes to void, they press down on the void switch 107. This will actuate the valve causing it to open and urine to flow out of the bladder and through the urethra. When the patient has completed the void, they press down on the void complete switch 108. If they do not press the void complete switch, the valve will automatically close after a preset time (e.g., 2 minutes). The screen 103 on the remote-control device optionally displays the current bladder pressure 104 and the time since last void 105. If the bladder pressure exceeds a defined pressure (preferably slightly below the threshold pressure), the remote-control device will alert the user letting them know that they must void or leakage will occur. In one embodiment, the alert will take the form of a 1-minute vibration and then sound an alarm through the speakers 102. The remote-control device is preferably of size and weight to easily be attached to a keyring 101.

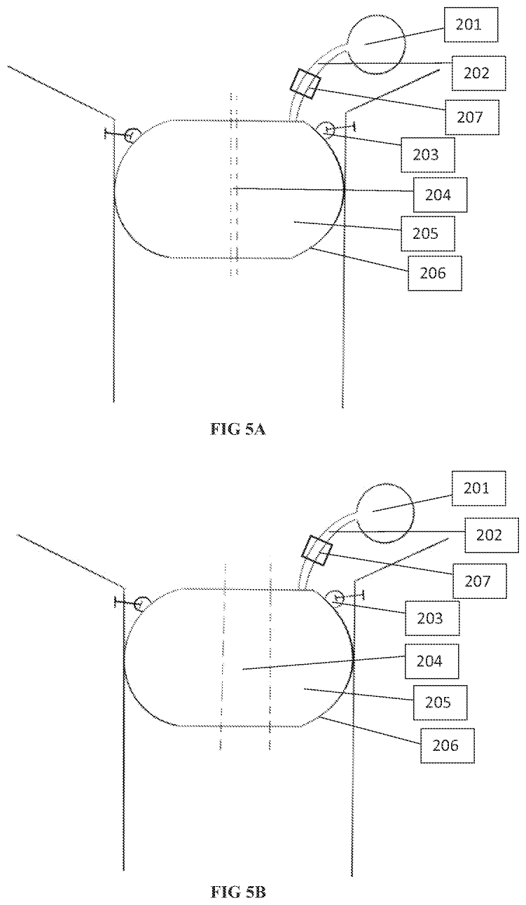

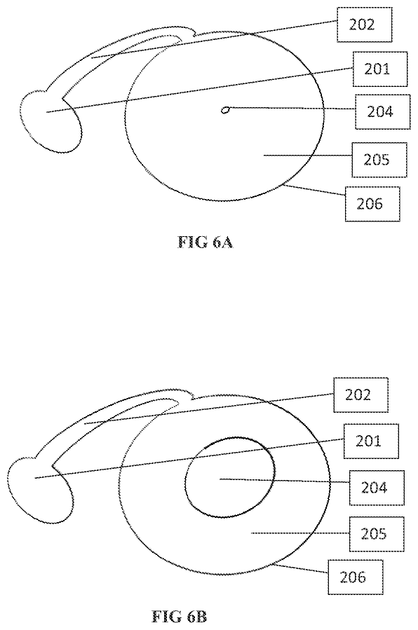

Referencing FIGS. 5A, 5B, 6A, 6B, 7A and 7B: in another embodiment of the valve device, the valve sac 205 material 206 forms a toroidal shape where the outer portion is filled with a working fluid and a bore 204 extends through it. The working fluid is transferred between the valve sac 205 and reservoir 201 thereby allowing the valve sac 205 to shrink and expand, i.e., open and close. This embodiment may also be positioned and anchored using anchors or sutures 203 in the bladder neck or bladder. The working fluid is filled and discharged from the valve sac 205 by a pump 207 along passage 202. Using an external remote-controller, the pump 207 between the valve sac 205 and reservoir 201 can be actuated causing fluid to move out of the valve sac 205 and into the reservoir 201, reducing the pressure in the outer portion of valve sac 205 and widening and opening the bore 204 to allow passage of urine. Once voiding is complete, the pump 207 can be actuated again in reverse, to cause fluid to move back into the valve sac 205, thereby closing 204 to prevent urine leakage from the urethra and bladder.

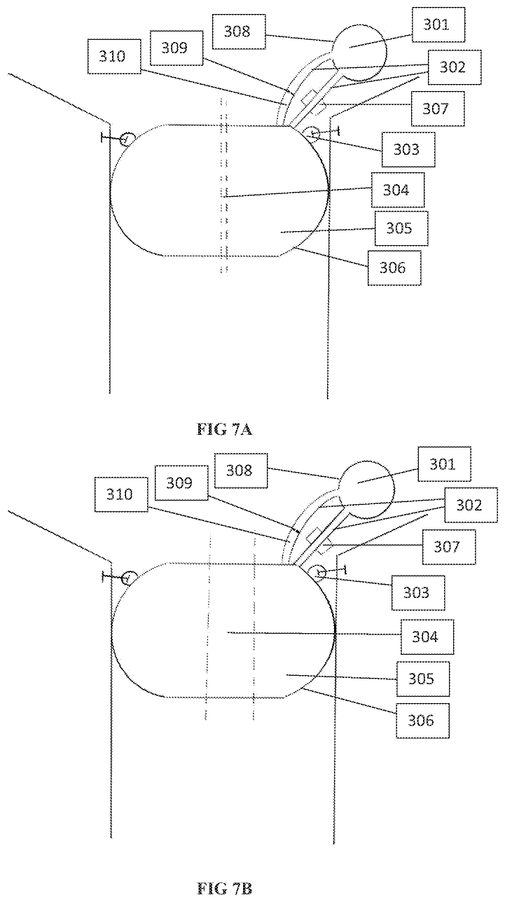

Referencing another embodiment of the valve device as shown in FIGS. 7A and 7B, the toroidal valve sac 305 and spherical reservoir 301 are respectively surrounded by elastic membranes 306 and 308, and the device is affixed in the urethra by anchoring of flanges 303. The tension in the elastic membrane surrounding the reservoir 301 is higher than that of the valve sac 305 because reservoir 301 is preferably formed of a more stretch resistant material than valve sac 305's elastomeric material. Additionally, there is a first tube 302 connecting the valve sac and reservoir, which contains a pump 307. The other tube 310 acts as a flow restrictor and optionally contains an orifice 309 to restrict flow.

In its rest state, the valve sac 305 is completely filled and the bore 304 at the center of the toroid is closed. In this state, no urine leakage occurs through the urethra. When the pump 307 is actuated by an external remote control, the fluid moves from the valve sac 305 into the reservoir 301 causing the bore 304 to open. The pump 307 continues to push fluid into the reservoir 301 until the void is complete. The pump's flow rate will be in excess of the leakage flow rate through the lumen 310. When the void is complete, the pump 307 shuts off and the fluid moves from the reservoir 301 back into the valve sac 305 through the lumen 310, under the excess pressure in the reservoir 301 caused by the fluidic stretching of its more stretch resistant material. The valve sac 305 is thereby closed and no urine leakage occurs. The geometry and tensile properties of the valve sac 305 and reservoir 301 are designed such that, in the equilibrium state, there is sufficient volume and pressure in the valve sac 305 to prevent urine leakage from the bladder.

In the embodiments depicted in FIGS. 5A to 7B, the shrinking and expanding of valve sac 205 or 305 can occur either at the hole of the toroid or at the seal between the toroid and urethra wall, or both. In one embodiment, to control where the shrinking and expansion occurs, the material properties in the zone of desired shrinking/expansion can be varied.

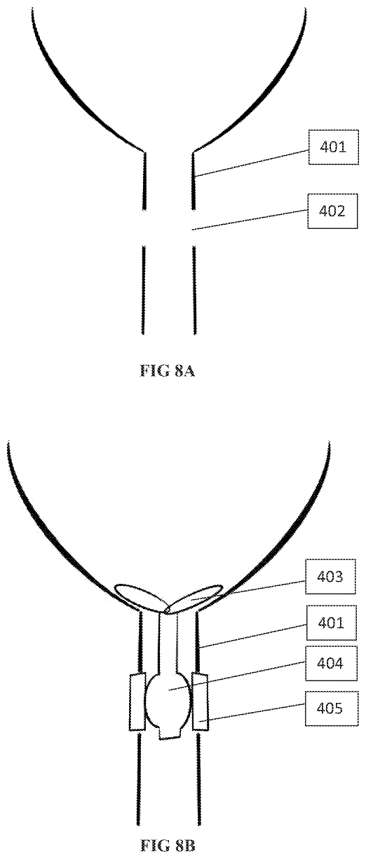

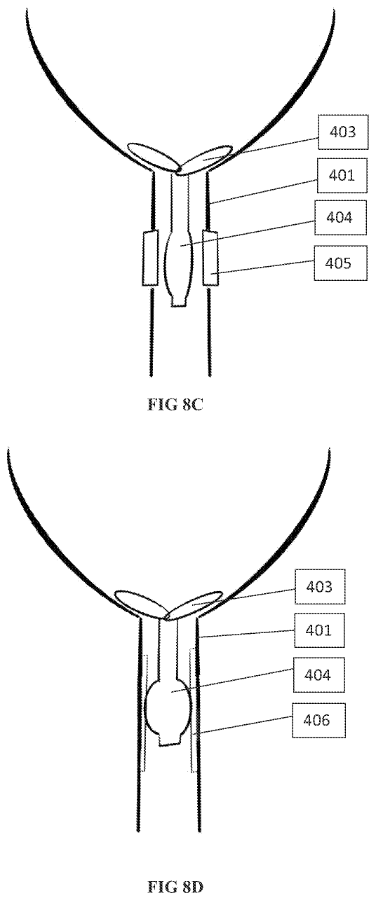

Another implantation embodiment for the valve is shown in FIGS. 8A-8D Here, a portion of the urethra 401 near incision 402 is cut away to allow implantation of a hollow tube 405 at the position which was cut. The hollow tube 405 may be sutured in place to act as a section of the urethra. A valve 404 (preferably of the type shown in FIGS. 1A to 1G) is placed to block urine flow by forming a seal with the hollow tube 405 instead of the urethral wall. Valve 404 may also be any of the embodiments described above (including the toroidal shapes). The seal against the urethral wall is improved by having a consistent surface (the inner side of tube 405) for the valve sac to mate with.

Alternatively, as in FIG. 8D, instead of cutting the urethra, a smaller diameter hollow tube 406, or a series of such smaller diameter hollow tubes 406 may be pushed into the urethra to lie in an appropriate position to seal against a valve device.

Referencing FIGS. 9A and 9B, an expanded view of the valve sac 10 from FIGS. 1C to 1G is shown, to better see the channels—503, which facilitate urine leakage when the bladder pressure exceeds the threshold pressure. In prototype experiments, it was observed that when the valve sac 10 had a smooth surface, there was the potential for the creation of a hermetic seal between the valve sac 10 and the urethra, such that the valve did not leak as intended at the threshold pressure. The channels 503 allow for the seal between the valve sac 10 and the urethra to be breached at the threshold pressure, thereby facilitating timely leakage before urine accumulation in the bladder causes backflow through the ureters to the kidneys.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation

What is claimed is:

1. A urethral valve comprising:
a valve sac formed of an elastomeric or viscoelastic material, wherein the valve is fixed within the bladder or the urethra so that the major portion of the valve sac resides in the urethra lumen and wherein the outer surface of the valve sac is adapted for sealing against the inner urethra lumen wall; and
an externally actuated elongation mechanism within the valve sac, having at least two portions which can separate when actuated so as to elongate the valve sac co-axially with the urethra lumen and thereby reduce outer diameter of the valve sac and break the seal against the urethra lumen wall.

2. The urethral valve of claim 1 wherein the elastomeric material encases a viscoelastic fluid.

3. The urethral valve of claim 1 wherein the elastomeric or viscoelastic material is sufficiently deformable to allow urine leakage around the outer surface of the valve sac when the urethral device is implanted and the urine pressure in the bladder passes a threshold.

4. The urethral valve of claim 1 wherein the valve sac has channels in its outer surface.

5. The urethral valve of claim 1 wherein the elastomeric material is a silicone material or a rubber or rubber-like material.

6. The urethral valve of claim 1 wherein the externally actuated elongation mechanism further includes a drive mechanism that converts rotational motion to linear motion in order to separate the two portions.

7. The urethral valve of claim 6 wherein drive mechanism includes a round gear or a screw and an electric motor to generate the rotational motion.

8. The urethral valve of claim 1 wherein the externally actuated elongation mechanism includes a solenoid.

9. The urethral valve of claim 1 further including wires, flaps, or flanges, attached to the elastomeric or viscoelastic material such that the valve can be affixed to tissue in the region of the bladder neck or urethra through the wires, flaps, or flanges.

10. The urethral valve of claim 1 wherein the viscoelastic material is an amorphous polymer, a semicrystalline polymer or a biopolymer.

11. The urethral valve of claim 1 further including at least one axial lumen positioned in the urethra to seal against the outer surface of the valve sac.

12. The urethral valve of claim 1 wherein the elongation mechanism is externally actuated by magnetic induction and/or attraction or repulsion.

13. The urethral valve of claim 2 further including an inner sac that isolates the fluid from the elongation mechanism.

14. A urethral valve comprising:
a toroidal valve sac formed of an elastomeric material and filled with a fluid, wherein the valve sac has a central bore, and is fixed within the bladder or urethra so as to block the urethra when the central bore is closed and permit urine passage when the central bore is open; and
an elastomeric reservoir sac in fluid communication with the valve sac and a pump which can be actuated to move the fluid between the valve sac and the reservoir, and wherein moving fluid into the reservoir opens the central bore and then moving the fluid back into the valve sac closes the central bore.

15. The urethral valve of claim 14 further including wires, flaps, or flanges attached to the elastomeric material such that the valve can be affixed to the bladder or urethra through the wires, flaps, or flanges.

16. The urethral valve of claim 14 further including at least one axial lumen affixed to the urethra in a position to seal against the outer surface of the valve sac.

17. The urethral valve of claim 14 wherein a primary lumen provides said fluid communication between the elastomeric reservoir sac and the valve sac and further including a secondary lumen connecting the valve sac and the reservoir sac, said secondary lumen containing an orifice.

18. The urethral valve of claim 17 wherein the elastomeric reservoir sac is formed of a more stretch resistant material than the valve sac elastomeric material.

19. The urethral valve of claim 14 wherein the elastomeric material or reservoir sac is a silicone material or a rubber or rubber-like material.

20. A urethral valve comprising:
a biocompatible device comprising of a proximal end and a distal end comprising:
a concentric assembly extending from the proximal end to the distal end of the biocompatible device, the concentric assembly comprising:
a concentrically hollow tube open at both ends to the urinary tract;
a concentric torque assembly comprising:
a torque mechanism at the proximal end; and
a threaded valve body connected to the torque mechanism and extending from the proximal to distal end wherein the threaded valve body is rotated to move a portion of the valve body towards the distal end
a biocompatible coating assembly comprising:
a biocompatible elastic coating that covers the entirety of the device laterally and from the proximal to distal ends; and
a valve sac located proximal to the lower half of the valve extension mechanism but distal to the anchors and comprising:
a viscoelastic material; and
an additional layer of biocompatible elastic coating that covers the entirety of the device latterly and from the proximal to distal ends; and the valve sac will be configured to:
the inner diameter of the urinary tract of a particular patient; and
allowed to leak to avoid over pressure in a patient's bladder;
an anchor system located distal to the torque mechanism and proximal to the valve sac extended laterally through a patient's bladder, comprised of the biocompatible elastic coating material, and configured to allow for sutures to be installed through the anchors.

21. A method of implanting the urinary valve device of claim 1 in a subject, the method comprising:
opening the urinary tract through a small incision;
opening the urinary bladder through a small incision;
placing the distal end of the urinary valve device through the urinary bladder opening; and
unfolding the anchor system of the urinary valve device; and
implanting urinary valve device using sutures passed through the anchors and the urinary bladder walls.

22. A method of draining a fluid from the bladder of the subject:
implanting the urinary valve device of claim 1 into the subject;
actuating the electromagnetic drive mechanism to open the urinary valve device;
allowing the fluid from bladder of the subject to evacuate; and
actuating the electromagnetic drive mechanism to close the urinary valve device.
